(19) United States
(12) Patent Application Publication
(10) Pub. No.: US 2025/0360295 A1
(43) Pub. Date: Nov. 27, 2025

(54) Adaptive, Multi-Injection Port, Double Balloon Catheter for Organ-Based Local Delivery of Gene Therapy

(71) Applicant: Wisconsin Alumni Research Foundation, Madison, WI (US)

(72) Inventors: John Booske, McFarland, WI (US); Paul Laeseke, Madison, WI (US); Susan Hagness, Madison, WI (US)

(21) Appl. No.: 18/669,699

(22) Filed: May 21, 2024

(51) Int. Cl. A61M 25/10; A61M 25/00

(52) U.S. Cl. CPC A61M 25/1011; A61M 25/005

(57) ABSTRACT

A “localizable” systemic gene therapy system is provided substantially increasing the transfection efficiency of the gene vectors into targeted tissue cells and substantially reducing the escape of the gene vectors from the targeted tissue volume, such as would waste the vectors, promote undesired immune reactions, and/or incur prohibitive costs for the required dose of gene-containing virus vectors. In this regard, the invention provides a means to simultaneously achieve local cell membrane permeability for virus vector transport and gene-containing vector injection in a portion of a vascularized organ. It includes a double-balloon catheter that create a finite contained volume in a blood vessel for the introduction of vectors with reduced loss along with a percutaneously inserted needle electrode providing increased cell membrane permeability for virus vector transport of the cells by creating an electric field in the same location where the vectors are injected.

### CROSS REFERENCE TO RELATED APPLICATION

### Background of the Invention

The present invention relates to a method and apparatus for drug or gene therapy, and more particularly, an improved drug or gene therapy delivery system using electrical stimulation to enhance uptake into cells.

Genetic mutation based metabolic diseases significantly reduce quality of life for hundreds of millions of people in the world and account for 70% of child hospitalizations and 10% of adult hospitalizations. There are hundreds of such diseases, including diabetes, cystic fibrosis, sickle cell anemia, hemophilia, and thalassemia. Many of them involve the liver due to its central role in metabolism.

Gene therapy is a promising treatment strategy for these diseases, potentially curing them by inserting functional genes (i.e., a functional portion of a DNA sequence) into a portion of cells within the target organ, for example, liver cells, thus correcting the inherited metabolic discrepancy. Only a small fraction of liver cells (hepatocytes) need to be converted, for example, about 5% in the liver, in order to produce therapeutic gene products sufficient to cure Type 1 diabetes. Gene therapy is also being actively investigated for curing diseases involving vascularized tissue mass bodies, e.g., cancer tumors.

A carrier, or vector, of genes or gene fragments, such as a virus capsid, can be used to deliver foreign, functional genes into cells. By transferring the functional gene into a virus that either enters the cell membrane through endocytosis (viruses without a lipid envelope) or binds to receptors on the cell membrane and fuses with the cell membrane thus releasing the genetic material (viruses with a lipid envelope), genes can be introduced into the cell. Depending on the virus used to deliver the gene, the viral genetic material either integrates into a chromosome of the cell or persists episomally without integration within the nucleus of the cell and expresses the introduced gene to treat the genetic defect.

Systemic gene therapy, which delivers functional genes via the circulatory system, has been found to be a successful delivery method for functional genes in small mammals (smaller than an average dog). However, this treatment has not been found to be scalable to large mammals for three reasons:

First, inefficient transduction of target cells necessitates large, cost-prohibitive gene vector doses. The larger size of the animal and more extensive blood flow pathways necessitates much larger doses of expensive vectors in order to convert the necessary amount of hepatocytes for effective therapy.

Second, the patient may have pre-existing antibodies that neutralize a virus capsid used as a gene vector rendering therapeutic attempt less effective or ineffective.

Third, systemic injection of such large quantities of the virus vector can trigger adaptive immunity that destroys not only the virus but also the genetically modified cells.

Compensating for these problems by introducing large amounts of vector is impractical because of the high expense of producing the vector and the inherent risks associated with injecting large amounts of virus into a human patient.

### SUMMARY OF THE INVENTION

The present invention provides a “localizable” liver gene therapy system substantially reducing the escape of the gene vectors from the tissue mass or organ, e.g., the liver, such that the waste of vector through systemic dilution is minimized, which would also limit the undesired immune reactions. In this regard, the invention describes a two inflatable balloon catheter that delivers a volume of vectors into a contained length of blood vessel to precisely deliver a high concentration of virus to the surrounding tissue. The high concentration of virus to a small, contained volume of tissue increases the uptake of vectors with reduced vector loss. While the contained length of vector delivery would seem to be counter to the intent of treating a large amount of tissue, parallel electrodes used in conjunction with vector delivery produce increased cell membrane permeability for viral vector transport in the tissue region between the electrodes offsetting this localization of vector delivery and improving uptake of the vector.

For example, a drug delivery catheter is inserted into a venous access site for hepatic vein catheterization. The medical professional may visualize the hepatic vein using computed tomography (CT) scan, ultrasound, or x-ray (fluoroscopy) guidance to advance the catheter into a blood vessel of the liver. A pair of inflated balloons flanking an active delivery section of the catheter may secure the location and position of the catheter's active delivery section. Gene therapy, e.g., viral vectors are then injected through the catheter to pass outward through egress holes in the active delivery section of the catheter to define a gene delivery area in the surrounding tissue.

Next, a separate minimally invasive needle electrode is inserted parallel to the catheter intravenously or through a blood vessel to create an electric field. A voltage relative to another electrode, e.g., located on the outside of the catheter wall, is delivered as a pulse to the needle electrode to create an electric field in the gene delivery area outside of the catheter. This results in an improved transduction rate of the viral vectors into the hepatic cells and therefore improved conversion of the hepatocytes with smaller vector doses.

The present invention provides a therapeutic delivery system comprising a catheter providing a distal end having a first and second inflatable balloon spaced apart along the distal end to define an intervening catheter section and at least one passageway through a delivery lumen of the intervening catheter section for the delivery of a therapeutic substance to a therapy site. A first electrical conductor is supported by and extends along the outside of the catheter along a first axis. A second electrical conductor insertable percutaneously through tissue of the therapy site extends substantially parallel to the intervening catheter section of the catheter along a second axis and is connected to a power supply delivering an electric voltage to the second electrical conductor relative to the first electrical conductor, configured to increase cell membrane permeability. The second electrical conductor includes an inner and outer coaxial conductive element and the outer coaxial conductive element is absent across from the first electrical conductor to allow for creation of an electrical field outside of the catheter, between the first and second electrical conductors.

It is thus a feature of at least one embodiment of the invention to use one or more thin needle electrodes inserted percutaneously near the liver (through tissue or within a vein) to create an electric field more precisely in the small drug delivery area to avoid adverse effects related to large vector doses.

The distance between the catheter and the second electrical conductor may be approximately 5 to 30 mm.

It is thus a feature of at least one embodiment of the invention to simplify the placement of the active electrode in close proximity to the drug delivery catheter without having to insert the active electrode through a blood vessel.

The first electrical conductor may be molded into a wall of the catheter and extend outside of the wall of the catheter. The first electrical conductor may be attached to the outer surface of the wall of the catheter. The first electrical conductor may form a helix.

It is thus a feature of at least one embodiment of the invention to use the catheter as the grounded return electrode without interfering with the delivery of therapeutics.

The first electrical conductor may be grounded.

It is thus a feature of at least one embodiment of the invention to use a separate needle electrode device to act as the active electrode which may be inserted around the grounded return electrode, simplifying the creation of an electric field in three dimensional space.

The second electrical conductor may be copper plated steel, stainless steel, chromium, tantalum, titanium, gold, platinum, nickel, zirconium, copper, and alloys of these metals, with an outer diameter less than 0.7 mm.

It is thus a feature of at least one embodiment of the invention to allow the thin, minimally invasive needle to be inserted into the tissue at a desired angle commensurate with the catheter angle independent of the position or angle of any blood vessels.

The equipotential lines of the electric field in a plane containing the first and second axes may be substantially evenly spaced and parallel between the first and the second electrical conductors.

It is thus a feature of at least one embodiment of the invention to create uniform electric fields which are equally applied to the cells of the drug delivery area to more accurately determine optimal electrical stimulation protocols.

A position of at least one of the first and second inflatable balloon may be movable along a length of the catheter.

It is thus a feature of at least one embodiment of the invention to adjust the length of the active delivery area of the catheter to accommodate variations in the organ or tissue delivery site and to adjust for different drug delivery applications using a standardized catheter assembly or kit of catheters of different lengths.

At least one therapeutic substance may comprise at least one of a drug or gene therapy.

It is thus a feature of at least one embodiment of the invention to optimize delivery of viral vector using electric field pulses of low duration to induce holes (increase permeability) in the cell membrane without affecting cell viability.

The present invention also provides a method for delivering one or more therapeutic substances to a patient, comprising: providing a catheter comprising a distal end having a first and second inflatable balloon spaced apart along the distal end to define an intervening catheter section and at least one passageway through a delivery lumen of the intervening catheter section for the delivery of at least one therapeutic substance to a therapy site, a proximal end having a therapeutic injection port, and a first electrical conductor extending along the catheter; providing a second electrical conductor insertable extending substantially parallel to the intervening catheter section of the catheter, wherein the second electrical conductor includes an outer coaxial conductive element and wherein the outer coaxial conductive element is absent from the second electrical conductor across from the intervening catheter section to allow for the creation of an electrical field across the first and second electrical conductors; inserting the catheter at a first insertion site into a blood vessel of a patient along a first axis; inserting the second electrical conductor at a second insertion site through tissue of the therapy site substantially parallel to the intervening catheter section of the catheter along a second axis; injecting the at least one therapeutic substance into the therapeutic injection port of the catheter and through the delivery lumen of the intervening catheter section to deliver the at least one therapeutic substance into surrounding cells; and delivering an electrical charge to the second electrode to produce a voltage across the first and second electrical conductors configured to increase cell membrane permeability of the surrounding cells.

It is thus a feature of at least one embodiment of the invention to separate the application of voltage to a thin minimally invasive needle electrode which may be easily inserted through tissue at various angles to more closely match the angle of the drug delivery catheter held within the blood vessel.

The second electrical conductor may be inserted approximately 5 to 30 mm from the catheter.

It is thus a feature of at least one embodiment of the invention to increase the strength of the electric field between electrodes at lower voltage to optimize cell membrane permeability.

The second electrical conductor may be inserted at approximately the same angle with respect to a longitudinal axis of the patient as an angle of the catheter with respect to the longitudinal axis of the patient.

It is thus a feature of at least one embodiment of the invention to produce a substantially uniform electric field between short linear sections or “stations” along the blood vessel.

The method may further produce an electric field wherein the equipotentials of the electric field in a plane containing the first and second axes are substantially evenly spaced and parallel between the first and the second electrical conductors.

It is thus a feature of at least one embodiment of the invention to accurately determine electric field strength to calculate electric stimulation protocols for cell membrane opening to minimize cell death.

The method may further provide moving a position of at least one of the first and second inflatable balloon to vary a length of the intervening catheter section.

It is thus a feature of at least one embodiment of the invention to treat the majority of the length of the organ or sub-organ by adjusting the active delivery section of the catheter.

The method may further provide removing the second electrical conductor from the second insertion site and inserting the second electrical conductor at a third insertion site through tissue substantially parallel to the intervening catheter section of the catheter.

It is thus a feature of at least one embodiment of the invention to apply voltage around the circumference of the catheter in order to create an electric field in the entire volume of the liver.

The present invention also provides a method for delivering one or more therapeutic substances to a patient, comprising: providing a catheter comprising a distal end having an intervening catheter section and at least one passageway through a delivery lumen of the intervening catheter section for the delivery of at least one therapeutic substance at a therapy site, and opposite a proximal end having a therapeutic injection port; providing a first electrical conductor extending along the catheter wherein the first electrical conductor includes an outer coaxial conductive element; providing a second electrical conductor extending substantially parallel to the first electrical conductor, wherein the second electrical conductor includes an outer coaxial conductive element; wherein the outer coaxial conductive element of the first and second electrical conductors is absent across from the intervening catheter section to allow for creation of an electrical field across the first and second electrical conductors; inserting the catheter at a first insertion site into a blood vessel of a patient along a first axis; inserting the first electrical conductor percutaneously at a second insertion site through tissue of the therapy site substantially parallel to the intervening catheter section of the catheter along a second axis; inserting the second electrical conductor percutaneously at a third insertion site through tissue of the therapy site substantially parallel to the intervening catheter section of the catheter along a third axis; injecting the at least one therapeutic substance into the therapeutic injection port of the catheter and through the delivery lumen of the intervening catheter section to deliver the at least one therapeutic substance into surrounding cells of the therapy site; and delivering an electrical charge to at least one of the first and second electrical conductor to produce a voltage across the first and second electrical conductors configured to increase cell membrane permeability of the surrounding cells of the therapy site.

It is thus a feature of at least one embodiment of the invention to use multiple percutaneous electrodes to produce an electric field around the drug therapy site (in three dimensional space) without sequentially removing needle electrodes.

These particular objects and advantages may apply to only some embodiments falling within the claims and thus do not define the scope of the invention.

### BRIEF DESCRIPTION OF THE DRAWINGS

FIG. 1 is a front view of a human receiving intravenous gene delivery through catheterization of a hepatic vein of the liver through alternative venous access sites of the body;

FIG. 2 is a perspective view through the liver of the human of FIG. 1 after insertion of a drug delivery catheter in the hepatic vein of the liver and a needle electrode inserted proximate to the catheter through tissue;

FIG. 3 is a side elevation view of the drug delivery catheter of FIG. 1 inserted within the hepatic vein and having an active delivery portion flanked by occlusion balloons and a grounded electrode extending on an exterior of the wall of the catheter to effectuate an electric field with an active electrode of the needle electrode (not shown);

FIG. 4 is a schematic view showing the drug delivery catheter inserted into a lower hepatic vein of the liver and providing dispersion of viral vectors from the inner lumen of the catheter into a gene delivery area while the needle electrode is inserted parallel to the catheter to create an electric field between the catheter and the needle electrode;

FIG. 5 is a cross section view of the needle electrode of FIG. 1 having a conductor with an unshielded portion to effectuate an electric field with the grounded electrode of the drug delivery catheter;

FIG. 6 is a chart showing a pattern of electrical field pulses delivered to the conductor of the needle electrode;

FIG. 7 is a flow chart showing the method steps of gene therapy according to the present invention; and

FIG. 8 is a schematic view of an alternative embodiment showing the drug delivery catheter being flanked by a pair of needle electrodes inserted in parallel to create an electric field between the needle electrodes and across the drug delivery area.

### DETAILED DESCRIPTION OF THE PREFERRED EMBODIMENT

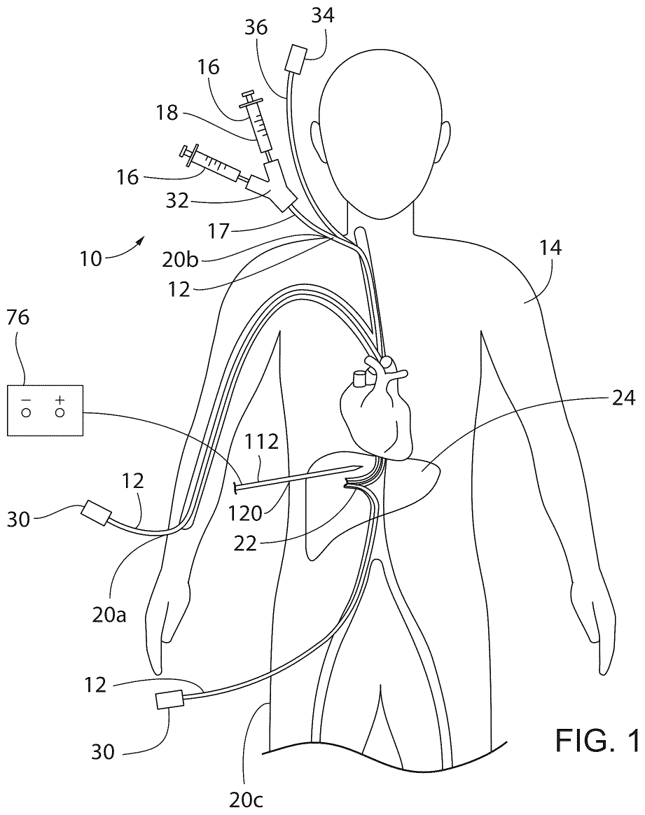

Referring now to FIG. 1, a gene therapy delivery system 10 may include at least one drug delivery catheter 12, and preferably one drug delivery catheter 12, inserted within the body to deliver fluids containing genes to a target organ of a human patient 14 for transduction into cells. The fluids may be intravenously injected by a syringe 16 or a pump (not shown) into a proximal end 17 of the drug delivery catheter 12 extending outside of the body and into a catheter insertion site. The fluids may include viral vectors 18, for example, retroviruses, lentiviruses, adenoviruses, adeno-associated viruses, and the like containing functional genes for gene therapy. Viral vectors 18 are generally larger than gene plasmids and therefore are more difficult to transport across cell membranes.

While the present invention is illustrated as a gene therapy delivery system 10, it is understood that the delivery system 10 may also be used to deliver drugs to, for example, tissue or tumor site.

The drug delivery catheter 12 may be inserted into a peripheral or central vein of the human patient 14 through a venous access site 20a-c allowing for catheterization of a hepatic vein 22 of the liver 24 of the human patient 14, for example, inserted at the antecubital vein 20a, the jugular vein 20b, or the femoral vein 20c, as seen as alternative insertion sites in FIG. 1. In one embodiment, the drug delivery catheter 12 may be inserted at an incision inside the neck of the human patient 14 proximate to the jugular vein 20b and then drawn downward through the hepatic vein 22 into the liver 24. The medical professional may use a guide wire (not shown) to facilitate placement of the drug delivery catheter 12 allowing the drug delivery catheter 12 to be installed over the guide wire after placement of the guide wire. This catheterization process may also be facilitated by real-time visualization by a medical professional through CT scan, ultrasound, or x-ray (fluoroscopy) guidance.

Referring also to FIG. 3, the drug delivery catheter 12 may include at least one proximal port 30, for example, two proximal ports 30 connected by a Y connector 32, allowing different fluids to be injected into the drug delivery catheter 12. The drug delivery catheter 12 may also provide a separate balloon inflation port 34 and balloon inflation tube 36 co-extending with and substantially parallel to the drug delivery catheter 12 to provide inflation of one or more balloons 38 of the drug delivery catheter 12, as further described below. The balloon inflation port 34 may also include a valve controlling flow through the tube 36 to inflate or deflate the balloons 38 as desired.

While it is shown that the drug delivery catheter 12 is installed into the hepatic vein 22 of the liver 24 of the human patient 14 for gene therapy, it is understood that the drug delivery catheter 12 may also catheterize other organs or tissues of the human patient 14 such as the kidney or pancreas.

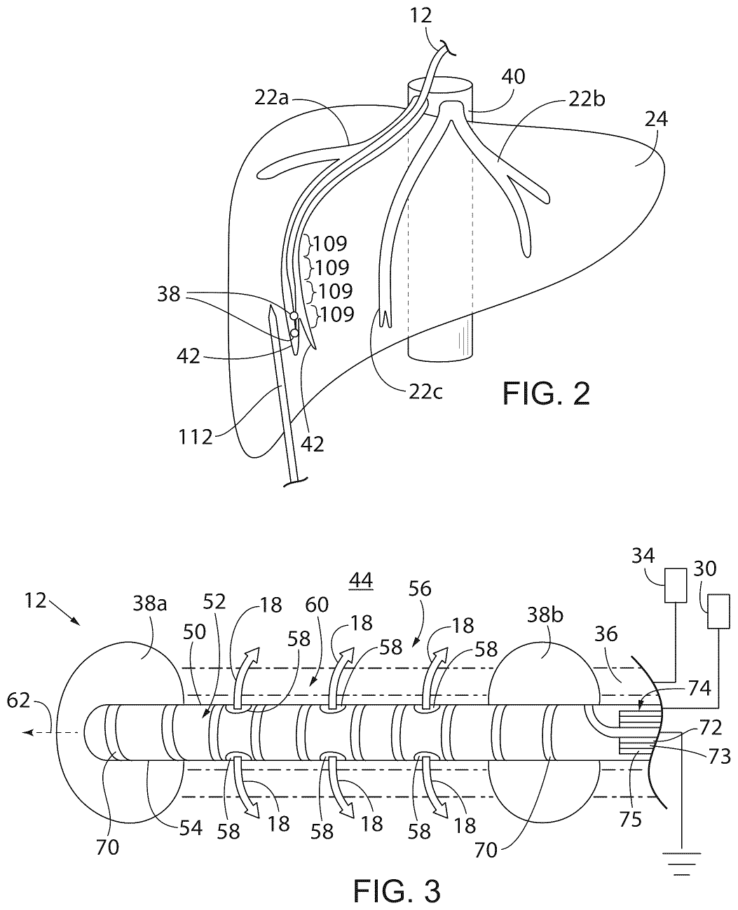

Referring now to FIG. 2, the drug delivery catheter 12 may be fed by the medical professional through the inferior vena cava 40 of the liver 24 and into one of the upper hepatic veins 22 of the liver 24, for example, the right hepatic vein 22a (as shown catheterized), left hepatic vein 22b, or middle hepatic vein 22c. The drug delivery catheter 12 may be further fed from the upper hepatic veins 22 into one of the lower blood vessels 42 branching from the upper hepatic veins 22 and which contact surrounding hepatic tissue cells 44 of the liver 24. For example, as shown, the drug delivery catheter 12 may be fed into one of the upper hepatic veins 22 and terminate in a lower blood vessel 42.

A separate fine needle electrode 112 may be fed into the liver 24 percutaneously by puncturing the abdominal skin at an insertion site 120 outside the liver 24 and advancing through liver tissue to extend substantially parallel to a section of the drug delivery catheter 12 within the lower blood vessel 42 to share adjacent tissue therebetween. In this manner, the drug delivery catheter 12 and needle electrode 112 are placed in relatively close proximity, about 5-30 mm or about 5-25 mm or about 5-20 mm or about 5-15 mm or about 5-10 mm, allowing a generally uniform electric field 46 to be created between the drug delivery catheter 12 and the needle electrode 112 as further discussed below.

Referring now to FIGS. 2 and 3, the drug delivery catheter 12 may have a construction facilitating dispersion of the viral vector 18 via the drug delivery catheter 12 as well as increase cell membrane permeability for viral vector transport of the hepatic cells 44 as described below.

The drug delivery catheter 12 may include a thin, flexible tube having an outer wall 50 made from a medical grade material such as vinyl, rubber latex, and silicone. The outer wall 50 has sufficient flexibility to flex with flexure of the drug delivery catheter 12 without holding a bent shape and without changing the stiffness of the outer wall 50. The outer wall 50 may have an outer diameter of 1.5-3 mm and an inner dimeter of 0.8-2.5 mm. The drug delivery catheter 12 may be consistent with a 5 French gauge catheter, 6 French gauge catheter, 7 French gauge catheter, 8 French gauge catheter or 9 French gauge catheter.

The drug delivery catheter 12 may come in varied lengths and outer diameters to accommodate the size of the receiving blood vessels and the distance between the access site 20a-c and the drug delivery site.

The outer wall 50 may provide an inner lumen 52 allowing for the flow of fluids therethrough. For example, the internal lumen 52 may allow for the passage of the viral vectors 18 from the proximal port 30 extending outside the body to a terminal end 54 of the drug delivery catheter 12 positioned within the lower blood vessel 42. The terminal end 54 may be a straight end terminating at a rounded enclosed tip or catheter cap. The terminal end 54 may also support a distal balloon 38a as further described below.

When installed within the lower blood vessel 42, a distal end 56 of the drug delivery catheter 12 may provide an active section 60 delivering the viral vector 18 out through the outer wall 50 and into the lower blood vessel 42 and further flanked by spaced apart balloons 38, as further discussed below. The outer wall 50 of the drug delivery catheter 12 may include one or more exit ports 58 within the active section 60 of the drug delivery catheter 12 allowing for the egress of viral vectors 18 injected into the drug delivery catheter 12, flowing through the inner lumen 52, and flowing outward into the surrounding hepatic cells 44. The exit ports 58 may be approximately 0.1-0.4 mm in diameter or may be approximately ¼ to ½ of the inner diameter of the drug delivery catheter 12. It is understood that any number of exit ports 58 may be included within the catheter outer wall 50 depending on the desired length of the active section 60, and in any configuration around a circumference of the outer wall 50.

The exit ports 58 may be linearly aligned along a longitudinal axis 62 of the drug delivery catheter 12, or alternatively, the exit ports 58 may be staggered in varying positions around a circumference of the outer wall 50 along the longitudinal axis 62 of the drug delivery catheter 12. The exit ports 58 may extend along substantially the entire length of the active section 60. For example, a single exit port 58 may be centered longitudinally within the active section 60 or more than one exit ports 58 may be spaced symmetrically about the center of the active section 60 and extend along substantially the entire length of the active section 60.

Alternatively, the outer wall 50 may be a porous material having microscopic holes allowing the viral vectors 18 to permeate through the outer wall 50 of the tube and disseminate into the surrounding hepatic cells 44.

Generally, it is understood that the active section 60 allows for the egress of the viral vectors 18 from the inner lumen 52 of the drug delivery catheter 12 into the hepatic tissue cells 44 surrounding the active section 60 of the drug delivery catheter 12.

The dispersion of viral vectors 18 volumes may be contained by at least two balloons 38, and preferably a pair of balloons 38a, 38b, spaced apart and flanking the active section 60 of the drug delivery catheter 12 delivering the viral vector 18. A distal balloon 38a may be positioned at or near the terminal end 54 of the drug delivery catheter 12 and a proximal balloon 38b may be positioned upstream from the terminal end 54 on the proximal side of the active section 60.

The balloons 38 may be integrally molded with the drug delivery catheter 12, for example, built within or as part of the outer wall 50 of the drug delivery catheter 12, or may be bonded to the outer wall 50 of the drug delivery catheter 12, for example, by a curable adhesive sealing an outer perimeter of the balloon 38 material to the outer wall 50 to create an airtight seal.

In certain embodiments, the distal balloon 38b and/or proximal balloon 38a may be movable or repositionable to allow the gene delivery area 64 bounded between the inflated balloons 38a, 38b to vary. In this respect, the distal balloon 38b and proximal balloon 38a may be repositionable along the length of the drug delivery catheter 12 and inflated to expand at various locations along the drug delivery catheter 12 in order to vary the gene delivery area 64 of the active section 60. Some of the proximal exit ports 58 may be “cut off” or removed from the active section 60 of the drug delivery catheter 12, for example, by moving the proximal balloon 38a toward the distal balloon 38b to prevent the egress of viral vectors 18 from exit ports 58 outside of the active section 60. Conversely, the number of proximal exit ports 58 may be increased by moving the proximal balloon 38a away from the distal balloon 38b to increase the number of exit ports 58 in the active section 60. Both the distal balloon 38b and proximal balloon 38a may be movable or repositionable, for example, to reposition the gene delivery area 64 along the length of the drug delivery catheter 12. Alternatively, the distal balloon 38b may be fixed in position at the terminal end 54 of the drug delivery catheter 12 while the proximal balloon 38a is moved to vary the length of the active section 60.

The balloons 38a may be separate from the drug delivery catheter 12 in order to be separately movable to desired locations along the longitudinal axis 62 and then expand out through longitudinally spaced openings in the outer wall 50 or co-expand with the outer wall 50 to hold the lower blood vessel 42.

The balloon 38 may be made of a material which resiliently deforms under radial pressure, for example, polyethylene (PE), nylon, polyamide, polyether block amides (PEBAX), polyethylene terephthalate (PET), silicone, POC, polypropylene, polyether block PBT and the like. The balloon 38 may include multiple layers and/or be coextruded and may also include additional fiber reinforcements. The size of the balloons 38 may vary depending on the size of the receiving lower blood vessel 42.

The pair of balloons 38a, 38b may be inflated simultaneously by injecting gas or liquid such as air, saline or iodinated contrast medium into the balloon inflation port 34 at the proximal end 17 of the drug delivery catheter 12 and through the balloon inflation tube 36 extending longitudinally with the drug delivery catheter 12. The balloon inflation tube 36 may be integrated with the drug delivery catheter 12, for example, molded into the outer wall 50 of the drug delivery catheter 12, bonded to the drug delivery catheter 12, or separate from the drug delivery catheter 12. It may be desired to include a separate balloon inflation tube 36 from the inner lumen 52 to independently control inflation or deflation of the balloons 38a, 38b while also using the inner lumen 52 as a fluid channel for the delivery of viral vectors 18. In this respect gas or liquid may flow through a balloon lumen that is separate from the inner lumen 52 of the drug delivery catheter 12. Alternatively, the gas or liquid may flow through the same tube as the inner lumen 52 of the drug delivery catheter 12.

The balloon 38 may be constructed as described above, and as described in U.S. Pat. Nos. 8,603,064 and 7,060,051, both of which are hereby incorporated by reference.

The balloons 38a, 38b may secure the positioning of the drug delivery catheter 12 within the lower blood vessel 42 by engaging the inner walls of the lower blood vessel 42 thus anchoring the drug delivery catheter 12 to the lower blood vessel 42 when inflated, and then deflated for removal of the drug delivery catheter 12 from the lower blood vessel 42 and from the body.

The balloons 38a, 38b also provide additional benefit by localizing the dispersion of the viral vectors 18 to a gene delivery area 64 substantially longitudinally bounded by the pair of inflated balloons 38a, 38b. In this respect, when viral vector 18 is injected into the inner lumen 52 of the drug delivery catheter 12, the inflated balloons 38a, 38b prevent the viral vector 18 from flowing downstream or upstream through the lower blood vessel 42. Therefore, the viral vectors 18 are encouraged to be absorbed into the nearby surrounding hepatic tissue, or may flow into smaller lateral vessels and capillaries to then be absorbed into the nearby surrounding hepatic tissue, instead of returning up the vein toward the proximal port 30 or down the vein from the terminal end 54 of the drug delivery catheter 12.

The inflated balloons 38a, 38b also block blood flow between the inflated balloons 38 during gene delivery.

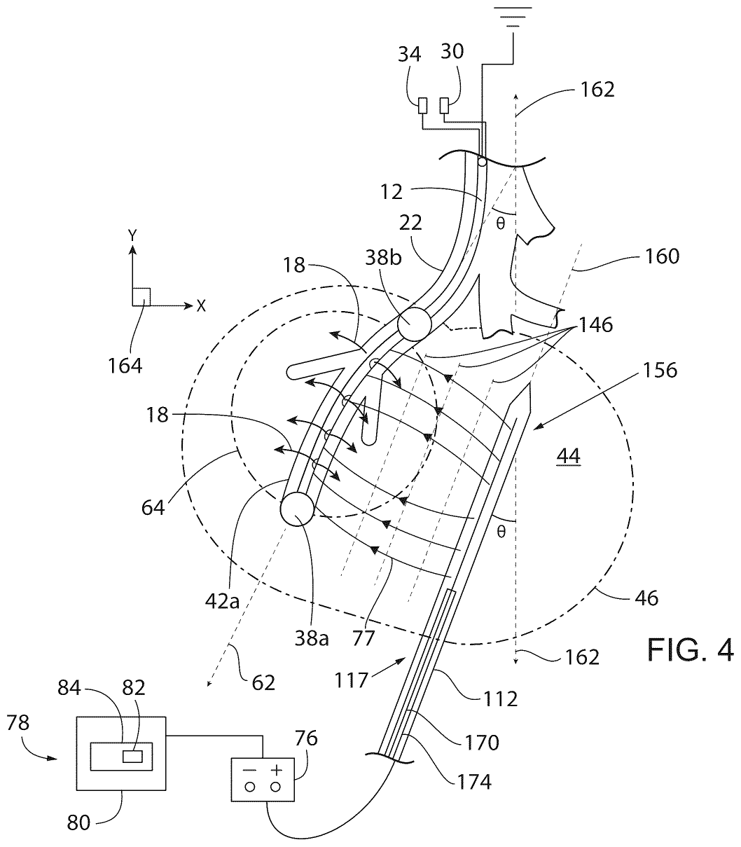

Referring to FIGS. 3 and 4, the drug delivery catheter 12 may support a grounded, return electrical conductor 70, extending coaxially within the internal lumen 52 along the longitudinal axis 62 of the drug delivery catheter 12. The electrical conductor 70 may also be coaxially positioned so that it does not block or obscure any of the exit ports 58, for example, it may be molded or partially molded into or onto the outer wall 50 of the drug delivery catheter 12 so that the electrical conductor 70 extends along the longitudinal axis 62 of the drug delivery catheter 12, for example, linearly or in a helix formation (as shown in FIG. 3). The outer dimension of the electrical conductor 70 is less than the thickness of the outer wall 50 of the drug delivery catheter 12 to be integrated into the outer wall 50 or may be attached to the inner or outer surface of the outer wall 50. The electrical conductor 70 may extend substantially an entire length of the drug delivery catheter 12, however, terminating before reaching the terminal end 54 of the drug delivery catheter 12, or before the distal balloon 38b.

The electrical conductor 70 may include one or more electrodes (contacting the surrounding tissue and fluid) extending to the exterior or outside of the outer wall 50. It is understood that any number of electrodes may be formed outside and along the drug delivery catheter 12. In a preferred embodiment, the electrical conductor 70 includes one or more electrodes molded or attached to the outer surface of the outer wall 50 to better achieve targeted electric field intensities with the least amount of voltage. It is understood that the electrical conductor 70 may be embedded within the outer wall 50 or positioned inside the outer wall 50 but include electrodes that extend to the outside of the outer wall 50 to receive current.

The electrical conductor 70 is configured to be grounded, or be at zero volts relative to the earth, and may be copper plated steel, stainless steel, chromium, tantalum, titanium, gold, platinum, nickel, zirconium, copper, alloys of these metals, and any other biocompatible electrically conducting metal, with or without an outer dielectric insulator permitting free passage of the electrical field but blocking electrical current flow and chemical reaction between the fluid and the material of the electrical conductor 70.

In an alternative embodiment, the electrical conductor 70 may be a return electrode connected to a power source to apply a voltage (i.e., potential difference) between the electrical conductor 70 and the needle electrode 112 as described further below. In this respect, the electrical conductor 70 may be at a negative voltage.

Optionally, the electrical conductor 70 may be shielded above the active section 60, or above the proximal balloon 38a, by an insulator 72 layer, an outer conductor 73 layer, and an insulator shield or jacket 75. The outer conductor 73 layer and the electrical conductor 70 are connected to ground potential. In this respect, the electric field is restricted to the dielectric and does not extend into the shielded section 74. For example, a shielded portion 74 may be a shielded cable, such as a coaxial cable, or a layered medical tubing with the electrical conductor 70 surrounded by a tubular insulating layer 72, for example, a solid plastic or a foam plastic such as solid polytetrafluoroethylene (PTFE) or solid polyethylene (PE) dielectric, and further surrounded by a tubular outer conductor 73, for example, a metal braided shield such as braided copper, aluminum or stainless steel wire, which may be plated or multi-layered, and may be further surrounded by an insulating shield or jacket 75, for example, a solid plastic such as polyvinyl chloride (PVC) which may be sealed around the outer conductor 73 to prevent reaction with fluids and the like.

The electrical conductor 70 may extend into the active section 60 with the insulator 72, outer conductor 73, and insulator jacket 75 removed. In this respect, the electric fields in the shielded portion 74 above the active section 60 are reduced and do not extend into the surrounding hepatic tissue cells 44 outside of the active section 60 of the drug delivery catheter 12. Alternatively, the electrical conductor 70 may remain insulated in the active section 60 with only the outer conductor 73 and insulator jacket 75 removed. In this respect, the electrical conductor 70 is not exposed to chemical reaction with the hepatic tissue cells 44. It is understood that the shielded portion 74 may still have an outer dimension less than the inner diameter of the outer wall 50 of the drug delivery catheter 12 to provide clearance therearound for flow of viral vector 18.

The drug delivery catheter 12 as described herein may include features of the two balloon catheter described in U.S. Pat. No. 10,918,861, also assigned to the present applicant, hereby incorporated by reference.

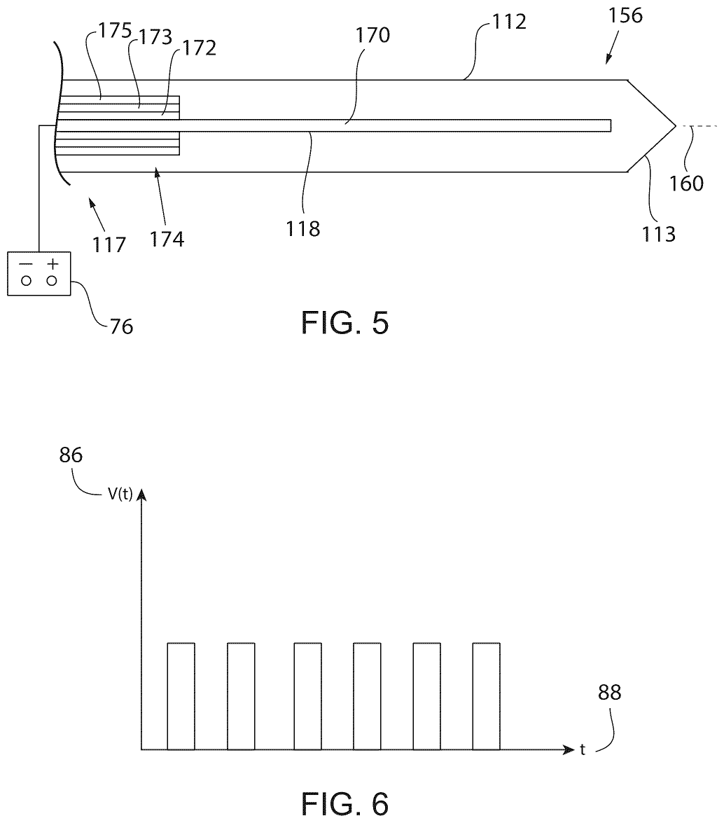

Referring to FIGS. 4 and 5, one or more separate needle electrodes 112 may be inserted into the hepatic tissue cells 44 such that it extends substantially parallel to the longitudinal axis 62 of the electrical conductor 70 and acts as the active electrode. The needle electrode 112 may support an antenna providing an active electrical conductor 170 having a length that is approximately the entire length of the needle electrode 112 but is shielded at the proximal end 117 by an outer dielectric insulator 118.

The active electrical conductor 170 may be a wire configured to carry electrical charge and may be copper plated steel, stainless steel, chromium, tantalum, titanium, gold, platinum, nickel, zirconium, copper, alloys of these metals, and any other biocompatible electrically conducting metal, with or without an outer dielectric insulator 118 permitting free passage of the electrical field but blocking electrical current flow and chemical reaction between the material of the active electrical conductor 170 and bodily fluid and surrounding tissue. The outer dielectric insulator 118 may be Teflon or another insulating material, as described below, shielding the active electrical conductor 170 at the proximal end 117 and exposing the active electrical conductor 170 at the distal end 156 to serve as the active electrode. The active electrical conductor 170 may be exposed to provide a single active electrode at the distal end 156 or an array of electrodes alternating with the insulating material.

In one embodiment, the proximal end 117 of the active electrical conductor 170 may be shielded by an insulator 172 layer, an outer conductor 173 layer, and an insulator shield or jacket 175. The outer conductor 173 layer may be connected to a ground potential while the active electrical conductor 170 is connected to a power source 76. In this respect, the electric field is restricted to the dielectric and does not extend from a shielded section 174. For example, the shielded portion 174 may be a shielded cable, such as a coaxial cable, or a layered medical tubing with the active electrical conductor 170 surrounded by a tubular insulating layer 172, for example, a solid plastic or a foam plastic such as solid polytetrafluoroethylene (PTFE) or solid polyethylene (PE) dielectric, and further surrounded by a tubular outer conductor 173, for example, a metal braided shield such as braided copper, aluminum or stainless steel wire, which may be plated or multi-layered, and may be further surrounded by an insulating shield or jacket 175, for example, a solid plastic such as polyvinyl chloride (PVC) which may be sealed around the outer conductor 173 to prevent reaction with fluids and the like.

The active electrical conductor 170 may extend across from the active section 60 of the drug delivery catheter 12 with the insulator 172, outer conductor 173, and insulator jacket 175 removed. In this respect, the electric fields are contained primarily to the length of the active section 60 of the coextending parallel drug delivery catheter 12. Alternatively, the active electrical conductor 170 may remain insulated in the active section 60 with only the outer conductor 173 and insulator jacket 175 removed. In this respect, electrical current is blocked from flowing into the surrounding hepatic tissue cells 44 and the active electrical conductor 170 is not exposed to chemical reaction with the hepatic tissue cells 44.

The needle electrode 112 has a beveled tip 113 allowing the sharp point to pierce the abdominal skin and be inserted percutaneously through the tissue mass of the liver 24.

In certain embodiments, the needle electrode 112 may be the active electrical conductor 170 surrounded by a hollow cannula. The hollow cannula may be made of medical grade plastic tubing made of stiff plastic medical grade material such as silicone, polyvinylchloride, polyethylene, and polyurethane. The hollow cannula has a beveled tip allowing the sharp point to pierce the abdominal skin. In this construction, the hollow cannula may be inserted through a vein of the liver 24 to be placed in close proximity, about 5-30 mm or about 5-25 mm or about 5-20 mm or about 5-15 mm or about 5-10 mm, to the drug delivery catheter 12. However, the hollow cannula does not need to be inserted coextensive with the drug delivery catheter 12, or at the venous access site 20a-c, or through the hepatic vein 22, but may be fed through a vein of the liver 24 to be positioned along the active section 60 of the drug delivery catheter 12.

In this respect, the present invention provides a needle electrode 112 (or hollow cannula) that may be easily inserted in parallel along the drug delivery catheter 12 either through tissue or a vein of the liver 24 without needing to catheterize the hepatic vein 22 coextensive with the drug delivery catheter 12.

The needle electrode 112 is thin and minimally invasive to facilitate precise placement of the electrode. The needle electrode 112 (or hollow cannula) has an outer diameter of approximately 0.1 to 1.5 mm or approximately 0.1 to 0.7 mm or approximately between 0.2 and 0.4 mm, and desirably approximately less than 1.5 mm or approximately less than 0.7 mm or approximately less than 0.4 mm to minimize discomfort to the patient.

Referring also to FIG. 6, the needle electrode 112 may be used in conjunction with a pulse generator 76 to provide a pulsed electrical charge to the active electrical conductor 170. For example, the pulse generator 76 may deliver a direct current (DC) 77 in the form of a repeated pulse or burst (e.g., pulse biased positive or negative) of an appropriate current amplitude and duration to the active electrical conductor 170 to create a voltage (i.e., potential difference) between the potential of the active electrical conductor 170 and the ground potential of the electrical conductor 70. An electric pulse may be applied to the active electrical conductor 170 receiving direct current 77 while the electrical conductor 70 acts as the return, grounded electrode.

As mentioned above, in an alternative embodiment, the electrical conductor 70 of the drug delivery catheter 12 may be connected to a power source and be at a negative voltage to act as the negative return electrode.

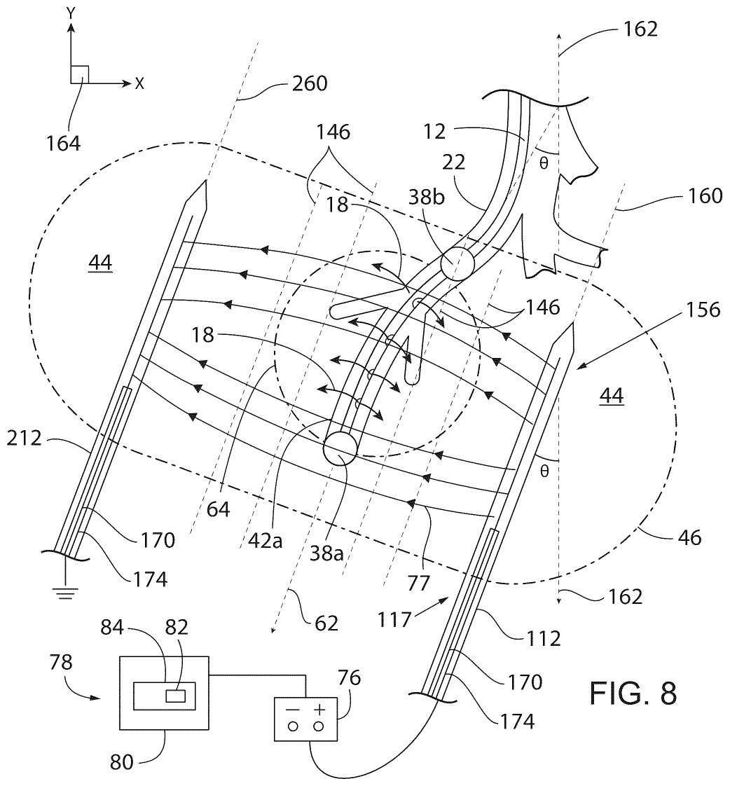

Turning briefly to FIG. 8, in an alternative embodiment, the electrical conductor 70 of the drug delivery catheter 12 may be omitted and replaced with a second needle electrode 212 with a similar construction as the needle electrode 112), and used in conjunction with the needle electrode 112, to act as the active or return electrode and create an electric field between the needle electrode 112 and the second needle electrode 212. The second needle electrode 212 may be connected to the pulse generator 76 to produce a pulsed electrical charge, e.g., acting as the active electrode or alternating with the needle electrode 112 as the active electrode and return electrode, or to create a ground potential when acting as the return electrode to the needle electrode 112 as shown.

The second needle electrode 212 is inserted along an axis 260 parallel to the axis 160. In this arrangement, the needle electrode 112 and second needle 212 may extend substantially in parallel to flank the gene delivery area 64 and thus create an electric field over the gene delivery area 64. The needle electrode 112 and second needle 212 may or may not be in parallel with the drug delivery catheter 12 while the needle electrode 112 and second needle electrode 212 are desirably in parallel to produce parallel and equidistant electric field equipotentials 146. The needle electrode 112 and second needle electrode 212 are in close proximity, about 5-30 mm or about 5-25 mm or about 5-20 mm or about 5-15 mm or about 5-10 mm in maximize the electric field strength therebetween.

Alternatively, the second needle electrode 212 could also be used with the electrical conductor 70 of the drug delivery catheter 12 and the needle electrode 112, acting as an additional active electrode to supplement the needle electrode 112 and making it easier to create an electric field in the three dimensional space of the gene delivery area 64 without removing the first needle electrode 112. Although two needle electrodes are shown in FIG. 8, it is understood that any number of additional needle electrodes, including one, two, three or more needle electrodes, may be used and inserted into hepatic cells 44 of the patient.

Pulse generators 76 suitable for in vivo cell membrane opening for viral vector transport as taught herein are sold by ECM under the trade name “830 square wave electroporation system”. Other pulse generators 76 are also commercially available and may be used with the present invention.

As illustrated in FIG. 5, the electric pulses may be repeated square pulses created by the pulse generator 76. It is understood that the electric pulses may take other shapes such as spikes or round waves. An electronic control circuit 78 may communicate with the pulse generator 76 to receive a preset voltage output and pulse length from the medical professional. The electronic control circuit 78 may hold, for example, a microprocessor 80 for executing a program 82 held in a stored memory 84.

The microprocessor 80 may also receive input data from the medical professional such as a distance between internal electrical conductor 70 and active electrical conductor 170, or a cross-sectional area between internal electrical conductor 70 and active electrical conductor 170, and execute the program 82 held in the stored memory 84 to output a voltage output 86 and pulse duration 88 to be used for effective pulse delivery, for example, using a lookup table. It is understood that internal electrical conductor 70 may be inserted along longitudinal axis 160 angled within the lower blood vessel 42 at an angle θ with respect to a longitudinal axis 162 of the human patient 14 and thus the needle electrode 112 will be inserted in a corresponding angle θ to be close to parallel with the active section 60 of the drug delivery catheter 12. Therefore, the distance between the electrical conductor 70 and the active electrical conductor 170 is the same along the active section 60.

The voltage output 86 may be selected so that the electric field 46 created between the internal electrical conductor70 and active electrical conductor 170 achieves or exceeds an efficacious electric field strength. For example, if the electric field 46 is too high, this can result in cell death through irreversible cell membrane opening. Alternatively, if the electric field strength is too low, the transmembrane potential required to permeabilize the cell membrane (typically 0.7 V) cannot be reached.

The pulse duration 88 may also be selected so that the electric field 46 created between the internal electrical conductor 70 and active electrical conductor 170 achieves or exceeds an efficacious electric field 46 strength. For example, if the pulse length is too short, (microseconds), the membrane capacitance may not charge up high enough to reach the required transmembrane potential. An efficacious electric field 46 strength may be coextensive with the gene delivery area 64.

It has been found that for gene delivery (compared to drug delivery), a combination of low electric field strength and long pulse length has been effective. For example, electric field intensities between 100-200 V/cm and pulse durations of 38-100 msec; and electric field intensities between 200-275 V/cm and pulse duration of about 50 msec have been found to be effective. Other parameters, which determine the efficacy of the delivery of viral vectors 18 into hepatic cells 44 are field strength, pulse length, shape of the pulse and number of pulses.

The electrical conductor 70 and active electrical conductor 170 are desirably parallel and equidistant so that the electric field equipotentials 146 in a plane 164 containing the longitudinal axis 62 of the delivery catheter 12 and longitudinal axis 160 of the needle electrode 112, are evenly spaced and parallel across the electrical conductor 70 and active electrical conductor 170 simplifying the determination of the electric field strength, pulse length, shape of the pulse and number of pulses.

The parameters used may be similar to those of electroporation described in Methods in Molecular Medicine, Vol. 37: Electrically Mediated Delivery of Molecules to Cells, Edited by: M. J. Jaroszeski, R. Heeller, and R. Gilbert, Humana Press, Inc., Totowa, NJ, and hereby incorporated by reference.

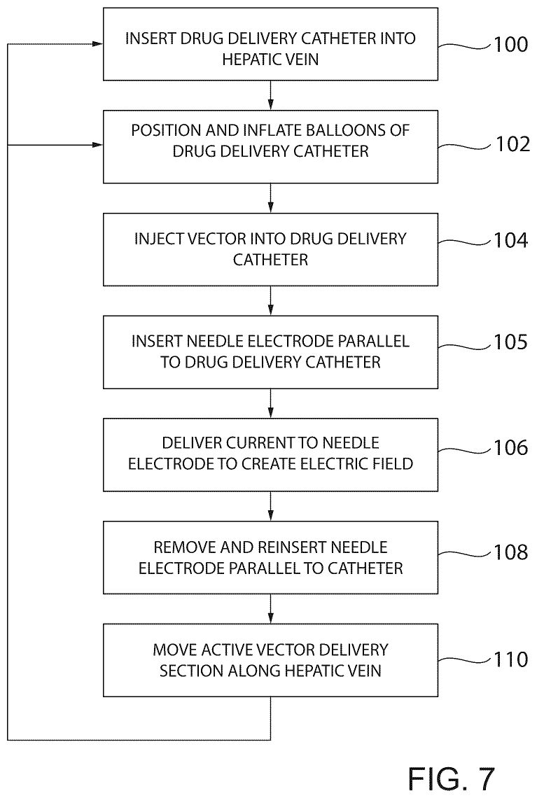

Referring now to FIG. 7, as noted above, the present invention uses a drug delivery catheter 12 of desired length and outer diameter inserted into the lower blood vessel 42 and a separate needle electrode 112 inserted along and parallel to the drug delivery catheter 12 to provide more efficient gene delivery eliminating the need for to catheterize neighboring blood vessels in close proximity. Such a procedure can use a single minimally invasive vascular catheter 12 catheterizing one or more lower blood vessels 42 of the liver 24 to perform direct gene delivery into the target liver with greater efficacy. To further enhance the efficiency of gene delivery for a large tissue area, the electrical conductor 70 of the catheter 12 and an active electrical conductor 170 of a separately inserted needle electrode 112 simultaneously increase cell membrane permeability for viral vector transport the hepatic cells 44 between adjacent electrodes to open temporary pores in the cell membrane allowing the vector 18 to readily enter the cells.

As indicated by process block 100, the drug delivery catheter 12 may be inserted through a venous access site 20a-c, for example, the jugular vein 20b, through a small incision at the neck of the human patient 14. The medical professional may use a guidewire and/or real-time visual imaging such as CT scan, ultrasound, or x-ray (fluoroscopy) to assist with the catheterization of the hepatic vein 22 of the right hepatic vein 22a.

As indicated by process block 102, once the catheter 12 is properly positioned within the lower blood vessel 42 of the right hepatic vein 22a of the liver 24 (desirably spanning the majority of the length of the liver), the balloons 38a, 38b may be repositioned to define an active section 60 delivering the viral vector 18 to a desired gene delivery area 64. The balloons 38 are inflated by injecting gas or fluid such as air, iodinated contrast or saline through the balloon inflation port 34 and through the inflation tube 36 in order to secure the catheter 12 in position, prevent further blood flow between the balloons 38a, 38b, and to isolate the gene delivery area 64. The balloons 38a, 38b of each drug delivery catheter 12 may inflate simultaneously and to a similar extent such that the inflated balloons 38a, 38b of each respective drug delivery catheter 12 are a similar size. In this respect the balloons 38a, 38b of the drug delivery catheter 12 may be a different size from the balloons 38a, 38b of the drug delivery catheter 12 to accommodate for different inner diameter sizes of the lower blood vessel 42.

Optionally, saline may be injected (retrograde flush) into the proximal ports 30 of the catheter 12 to wash the inner lumen 52 and gene delivery area 64 from obstructive blood and tissues or antibodies.

Then, as indicated by process block 104, the viral vector 18 may be injected (retrograde flush) into the proximal ports 30 of the catheter 12 in order to fill the inner lumen 52 of the catheter 12 with the viral vector 18 and to disseminate the vector 18 through the exit ports 58 of the outer wall 50 of the catheter 12, and further through the vascular walls of the lower blood vessels 42, and into the gene delivery area 64 surrounding the catheter 12. It is understood that the gene delivery area 64 may comprise of regions emanating from and surrounding the active section 60 of the drug delivery catheter 12.

Then, as indicated by process block 105, the needle electrode 112 is precisely inserted percutaneously through liver tissue or a vein of the liver 24 using visual imaging such as CT scan, ultrasound, or x-ray to extend parallel to the active section 60 of the drug delivery catheter 12 and approximately 5-30 mm away from the active section 60 of the drug delivery catheter 12 to create an electric field 46 in the area between and around the needle electrode 112 and drug delivery catheter 12 overlapping with the gene delivery area 64. In an alternative embodiment, if a second needle electrode 212 is inserted percutaneously through liver tissue or a vein of the liver 24 as seen in FIG. 8, the electric field 46 is created in the area between the needle electrode 112 and the second needle electrode 212 overlapping with the gene delivery area 64.

As indicated by process block 106, the medical professional may consider the location and distance between the catheter 12 and needle electrode 112 using visual imaging such as CT scan, ultrasound, or x-ray to determine a desired voltage output 86 and pulse duration 88 to be outputted by the pulse generator 76. The medical professional may program the pulse generator 76 to elicit a direct current 77 in short pulses, for example, square waves as illustrated in FIG. 6, to the needle electrode 112 to therefore create an electric field 46 between and surrounding the catheter 12 and the needle electrode 112. It is understood that the electric field 46 may comprise of a region surrounding the drug delivery catheter 12 and the needle electrode 112 and primarily extending across the drug delivery catheter 12 and needle electrode 112, the tissue area coextensive or greater than the gene delivery area 64.

Next, as indicated by process block 108, the needle electrode 112 may be repositioned, or a second needle electrode 212 inserted and positioned, radially around the active section 60 of the drug delivery catheter 12 and process block 106 repeated one or more times to further create a uniform electric field around the circumference of the drug delivery catheter 12 and to create an electric field in the three-dimensional space of the gene delivery area 64 of the liver.

Furthermore, as indicated by process block 110, the drug delivery catheter 12 may be withdrawn (or advanced) within the lower blood vessel 42 in order to repeat the drug delivery steps 102, 104 and electrical stimulation steps 105 and 106 at various longitudinal stations 109, as seen in FIG. 2, along the length of the lower blood vessel 42. Alternatively, the balloons 38a, 38b may be repositioned to define a new active section 60 at the different stations 109. Since the blood vessels are generally non-linear over an extended length, the drug delivery step 104 and electrical stimulation steps 105, 106, 108 may be reperformed at short longitudinal stations 109 along the length of the blood vessels to ensure a substantially uniform electric field is applied to the new gene delivery area 64. In this respect, the needle electrode 112 is also repositioned parallel to the new active section 60 at the different stations 109.

Next, as indicated by returning to process block 100, the drug delivery catheter 12 may be removed from the lower blood vessel 42 of the right hepatic vein 22a, and the drug delivery catheter 12 may be withdrawn and inserted into a lower blood vessel 42 of the middle hepatic vein 22b in a similar manner as described in process blocks 100 and 102. The drug delivery steps 102, 104 and electrical stimulation steps 105, 106, 108 may also be repeated in the new gene delivery area 64 at the new hepatic vein location. These steps may also be repeated for a lower blood vessel 42 of the left hepatic vein 22c to provide treatment over substantially the entire liver.

The increase of cell membrane permeability for viral vector transport of the gene delivery area 64 provided by the electric field 46 allows for the hepatic cells 44 in the gene delivery area 64 to be more susceptible to viral intake. In this respect, the catheter 12 and needle electrode 112 allows for simultaneous or nearly simultaneous delivery of genes to cells with increased cell membrane permeability for viral vector transport of the cells extending between the drug delivery catheter 12 and needle electrode 112. It is contemplated that the drug delivery described above may be performed once or may be conducted as repeated treatments as necessary.

It is understood that the gene therapy delivery system 10 may be performed on any mammal and of any size, for example, both small and large mammals.

It is understood that “drug”, “gene”, “viral vector”, and “vector” are used interchangeably, and “drug therapy” and “gene therapy” are used interchangeably to describe the delivery of therapeutic substances which are not intended to be limiting.

It is understood that “transduction”, “transducing”, “transfection”, and “transfecting” are used interchangeably to describe the insertion of genes or DNA into cells and are not intended to be limiting.

It is understood that “tissue”, “tissue mass”, “tissue cells” “organ”, “liver”, “tumor”, “tumor site” are used interchangeably to describe the target or site within the body receiving gene therapy and are not intended to be limiting.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A therapeutic delivery system comprising:
a catheter providing a distal end having a first and second inflatable balloon spaced apart along the distal end to define an intervening catheter section and at least one passageway through a delivery lumen of the intervening catheter section for the delivery of a therapeutic substance to a therapy site;
a first electrical conductor supported by and extending along the catheter along a first axis; and
a second electrical conductor insertable percutaneously through tissue of the therapy site to extend substantially parallel to the intervening catheter section of the catheter along a second axis and connected to a power supply delivering an electric voltage to the second electrical conductor relative to the first electrical conductor, configured to increased cell membrane permeability;
wherein the second electrical conductor includes an inner and outer coaxial conductive element and wherein the outer coaxial conductive element is absent across from the first electrical conductor to allow for creation of an electrical field outside of the catheter, between the first and second electrical conductor.

2. The therapeutic delivery system of claim 1, wherein a distance between the catheter and the second electrical conductor is approximately 5 to 30 mm.

3. The therapeutic delivery system of claim 1, wherein the first electrical conductor is molded into a wall of the catheter and extended outside of the catheter.

4. The therapeutic delivery system of claim 3, wherein the first electrical conductor forms a helix.

5. The therapeutic delivery system of claim 1, wherein the first electrical conductor is grounded.

6. The therapeutic delivery system of claim 1, wherein the second electrical conductor has an outer diameter less than 1.5 mm.

7. The therapeutic delivery system of claim 1, wherein equipotentials of the electric field in a plane containing the first and second axes are substantially evenly spaced between the first and the second electrical conductors.

8. The therapeutic delivery system of claim 1, wherein equipotentials of the electric field in a plane containing the first and second axes are substantially parallel between the first and the second electrical conductors.

9. The therapeutic delivery system of claim 1, wherein a position of at least one of the first and second inflatable balloon may be movable along a length of the catheter.

10. The therapeutic delivery system of claim 1, wherein at least one therapeutic substance comprises at least one of a drug or gene therapy.

11. A method for delivering one or more therapeutic substance to a patient, comprising:
providing a catheter comprising a distal end having a first and second inflatable balloon spaced apart along the distal end to define an intervening catheter section and at least one passageway through a delivery lumen of the intervening catheter section for the delivery of at least one therapeutic substance at a therapy site, and opposite a proximal end having a therapeutic injection port, and a first electrical conductor extending along the catheter;
providing a second electrical conductor extending substantially parallel to the intervening catheter section of the catheter, wherein the second electrical conductor includes an outer coaxial conductive element and wherein the outer coaxial conductive element of the second electrical conductor is absent across from the intervening catheter section to allow for creation of an electrical field across the first and second electrical conductors;
inserting the catheter at a first insertion site into a blood vessel of a patient along a first axis;
inserting the second electrical conductor percutaneously at a second insertion site through tissue of the therapy site substantially parallel to the intervening catheter section of the catheter along a second axis;
injecting the at least one therapeutic substance into the therapeutic injection port of the catheter and through the delivery lumen of the intervening catheter section to deliver the at least one therapeutic substance into surrounding cells of the therapy site; and
delivering an electrical charge to the second electrical conductor to produce a voltage across the first and second electrical conductors configured to increase cell membrane permeability of the surrounding cells of the therapy site.

12. The method of claim 11, further comprising inserting the second electrical conductor approximately 5 to 30 mm from the catheter.

13. The method of claim 11, wherein the second electrical conductor is inserted at approximately the same angle with respect to a longitudinal axis of the patient as an angle of the catheter with respect to the longitudinal axis of the patient.

14. The method of claim 11, further comprising producing an electric field in a plane containing the first and second axes wherein equipotentials of the electric field are substantially evenly spaced between the first and the second electrical conductors.

15. The method of claim 11, further comprising producing an electric field wherein equipotentials of the electric field in a plane containing the first and second axes are substantially parallel between the first and the second electrical conductors.

16. The method of claim 11, further comprising moving a position of at least one of the first and second inflatable balloon to vary a length of the intervening catheter section.

17. The method of claim 11, further comprising
removing the second electrical conductor from the second insertion site; and
inserting the second electrical conductor at a third insertion site through tissue substantially parallel to the intervening catheter section of the catheter.

18. The method of claim 11, further comprising grounding the first electrical conductor.

19. The method of claim 11, wherein the blood vessel is located within a vascularized organ, tissue, or tumor.

20. A method for delivering one or more therapeutic substance to a patient, comprising:
providing a catheter comprising a distal end having an intervening catheter section and at least one passageway through a delivery lumen of the intervening catheter section for the delivery of at least one therapeutic substance at a therapy site, and opposite a proximal end having a therapeutic injection port;
providing a first electrical conductor extending along the catheter wherein the first electrical conductor includes an outer coaxial conductive element;
providing a second electrical conductor extending substantially parallel to the first electrical conductor, wherein the second electrical conductor includes an outer coaxial conductive element;
wherein the outer coaxial conductive element of the first and second electrical conductors is absent across from the intervening catheter section to allow for creation of an electrical field across the first and second electrical conductors;
inserting the catheter at a first insertion site into a blood vessel of a patient along a first axis;
inserting the first electrical conductor percutaneously at a second insertion site through tissue of the therapy site substantially parallel to the intervening catheter section of the catheter along a second axis;
inserting the second electrical conductor percutaneously at a third insertion site through tissue of the therapy site substantially parallel to the intervening catheter section of the catheter along a third axis;
injecting the at least one therapeutic substance into the therapeutic injection port of the catheter and through the delivery lumen of the intervening catheter section to deliver the at least one therapeutic substance into surrounding cells of the therapy site; and
delivering an electrical charge to at least one of the first and second electrical conductor to produce a voltage across the first and second electrical conductors configured to increase cell membrane permeability of the surrounding cells of the therapy site.
